(19) United States
(12) Patent Application Publication
(10) Pub. No.: US 2002/0127544 A1
(43) Pub. Date: Sep. 12, 2002

(54) Modulators of US 28

(75) Inventors: Thomas J. Schall, Palo Alto, CA (US); Brian E. McMaster, Mountain View, CA (US); Daniel J. Dairaghi, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ChemoCentryx, San Carlos, CA

(21) Appl. No.: 09/944,163

(22) Filed: Aug. 30, 2001

Related U.S. Application Data
(60) Provisional application No. 60/228,974, filed on Aug. 30, 2000.
(60) Provisional application No. 60/229,191, filed on Aug. 30, 2000.
(60) Provisional application No. 60/229,365, filed on Aug. 30, 2000.

(51) Int. Cl. C12Q 1/70; A61K 31/519; A61K 31/498; A61K 31/473

(52) U.S. Cl. 435/5; 514/250; 514/267; 514/291

(57) ABSTRACT

Assays, compositions and methods of treatment are provided for modulating the binding of chemokines to US28 on the surface of cells.

### CROSS-REFERENCES TO RELATED APPLICATIONS

This application claims the benefit of U.S. Provisional Patent Application Serial No. 60/228,974, filed Aug. 30, 2000, and U.S. Provisional Patent Application Serial No. ______, filed Aug. 30, 2001, entitled “Bicyclic Compounds as Inhibitors of Chemokine Binding to US 28” (Attorney Docket No. 019934-001000US), the disclosures of each being incorporated herein by reference. Related subject matter is described in co-owned applications Ser. No. ______, filed Aug. 30, 2001, entitled “Reagents and Methods for the Diagnosis of CMV Dissemination” (Attorney Docket No. 019934-000910US/PCT) which claims the benefit of Ser. No. 60/229,191 filed Aug. 30, 2000; and in Ser. No. ______, filed Aug. 30, 2001, entitled “Inhibition of CMV Infection and Dissemination” (Attorney Docket No. 019934-002510US/PCT) which claims the benefit of Ser. No. 60/229,365, filed Aug. 30, 2000, the disclosures of each being incorporated herein by reference.

### STATEMENT AS TO RIGHTS TO INVENTIONS MADE UNDER FEDERALLY SPONSORED RESEARCH AND DEVELOPMENT

Not applicable

### BACKGROUND OF THE INVENTION

Cytomegalovirus (CMV) is an important human pathogen and a major opportunist which emerges to cause disease in the immuno-compromised such as AIDS patients, neonates, and individuals who have been given immunosuppressive drugs as part of a transplantation regimen. In these individuals, the consequences of CMV in acute or re-emerging infections can be dire, including retinitis, encephalitis, and pneumocystis, among other pathologies. Furthermore, in immuno-competent hosts, CMV establishes a persistent lifelong infection through which it has been linked to a variety of inflammatory conditions including coronary artery occlusion following heart transplant and arthrectomy and restenosis following angioplasty. CMV interacts with leukocytes during acute infection of the host as well as during lifelong latency. As such, leukocytes are important players in CMV-induced disease and have been implicated in the acute phase of infection as vehicles for dissemination of virus and as sites of residence during lifelong latency.

### SUMMARY OF THE INVENTION

In one aspect, the present invention provides an assay for identifying a compound useful for blocking CMV dissemination in a host by determining whether the compound inhibits the binding of a chemokine to US28 or a US28 fragment. Typically, the assay will be run as a competitive binding assay using a labeled chemokine. A variety of chemokines are known to bind to US28 and are useful in this aspect of the invention. Preferably, the chemokine is fractalkine and the assay is a radioligand binding assay.

In another aspect, the present invention provides methods for blocking CMV dissemination in a host by administering to the host an effective amount of a compound which blocks the binding of a chemokine to US28. Preferably, the compound is one which was identified using an assay of the present invention.

In yet another aspect, the present invention provides pharmaceutical compositions for the treatment of CMV comprising compounds identified in the present assays and further described below.

### BRIEF DESCRIPTION OF THE DRAWINGS

FIG. 1 illustrates the specific displacement of chemokine (fractalkine) binding to the US28 chemokine receptor.

FIG. 2 illustrates the signaling profile and cross desensitization between methiothepin and a chemokine ligand (fractalkine) for US28.

### DETAILED DESCRIPTION OF THE INVENTION

Abbreviations and Definitions

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. When used alone, the term “alkyl” refers to unsubstituted versions of the radicals indicated above. Substituted forms of “alkyl” are defined in more detail below.

The term “alkylene” by itself or as part of another substituent means a divalent radical derived from an alkane, as exemplified by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—S(O)₂—CH₃, —CH═CH—O—CH₃, —Si(CH₃)₃, —CH₂—CH═N—OCH₃, and —CH═CH—N (CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified by —CH₂—CH₂—S—CH₂CH₂—and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl or heterocyclyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “(C₁- C₄)haloalkyl” is mean to include trifluoromethyl, 2,2,2-trifluoroethyl, 4-chlorobutyl, 3-bromopropyl, and the like.

The term “acyl” is used in its conventional sense and refers to an organic radical derived from an organic acid by the removal of the hydroxyl group. Examples of “acyl” groups include acetyl, propionyl, butanoyl, hexanoyl, isobutyryl, octanoyl, and the like.

The term “aryl” means, unless otherwise stated, a polyunsaturated, typically aromatic, hydrocarbon substituent which can be a single ring or multiple rings (up to three rings) which are fused together or linked covalently. The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, ³-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy) propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from:—OR′, ═O, ═NR′, ═N—OR′, —NR′R″, SR′, —halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂) ═NR′,—S(O)R′, —S(O)₂R′, —S(O)₂NR′R″, —CN and —NO₂ in a number ranging from zero to (2m′+1), where m′ is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₈) alkyl, and (unsubstituted aryl)oxy-(C₁-C₄)alkyl.

Two of the substituents on adjacent atoms of the aryl or heteroaryl ring may optionally be replaced with a substituent of the formula —T—C(O)—(CH₂)q—U—, wherein T and U are independently —NH—, —O—, —CH₂—or a single bond, and q is an integer of from 0 to 2. Alternatively, two of the substituents on adjacent atoms of the aryl or heteroaryl ring may optionally be replaced with a substituent of the formula —A—(CH₂)r—B—, wherein A and B are independently —CH₂—, —O—, —NH—, —S—, —S(O)—, —S(O)₂—, —S(O)₂NR′—or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′—and —S(O)₂NR′—is selected from hydrogen or unsubstituted (C₁-C₆)alkyl.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14(¹⁴C). All isotopic variations of the compounds of the present invention, whether radioactive or not, are intended to be encompassed within the scope of the present invention.

General

CMV harbors in its genome an open reading frame (ORF), designated US28, which encodes a protein that acts as a functional receptor for certain human and viral chemokines. Upon infection of a cell by CMV, US28 is expressed on the surface of the infected cell and becomes capable of responding to chemokines in the environment. Because the virus on its own is inherently non-motile, and because chemokines and their receptors encoded by human cells are known to regulate the migration of leukocytes and other cells through the body, CMV US28 is now thought to be encoded by the virus to facilitate the dissemination of CMV through the body during and after infection. Therefore, agents which block the binding of chemokines to US28 are expected to be useful in inhibiting viral dissemination during acute or re-emerging CMV infection.

CMV US28 has been shown to bind a variety of human, murine, and virus-encoded CC chemokines in a variety of assay formats. In addition, the CX3C chemokine, Fractalkine, binds with a very high affinity (K_{I}˜50 pM) to US28. Fractalkine is expressed on certain endothelial cell surfaces and on populations of dendritic cells (DC), and may thus define a portal through which CMV infected cells go from the circulation to the tissue space, as well as find residence in the DC.

Since the US28 receptor is expressed on cytomegalovirus infected cells, and also in view of its ability to bind multiple chemokines, a small molecule inhibitor for this receptor would have significant use as an anti-CMV agent.

Accordingly, the present invention provides a novel mechanism for control of cytomegalovirus induced disease. By inhibiting dissemination of virus from sites of primary or recurrent infection, the compounds described herein can limit the viral spread to secondary organs and so limit viral replication. Unlike current herpes antiviral agents, the compounds described herein do not act at the stage of viral DNA replication and so are less prone to problems with toxicity and the development of viral resistance. Other GPCR targeted therapeutics have demonstrated high efficacy and been well tolerated for a number of indications.

Description of the Embodiments

A. Assays for Identifying Compounds Which Block Viral Dissemination

In one aspect, the present invention provides assays for identifying a compound capable of blocking CMV dissemination in a host, by determining whether the compound inhibits the binding of a chemokine to US28 or a US28 fragment.

The assays provided herein are typically cell-based assays in which a cell which stably expresses US28 is treated with a candidate compound and a chemokine in a competitive binding format. A variety of other assay formats are also useful in the present invention. For example, substrate-bound or support-bound chemokines (or ligands) can be contacted with a labeled cell or liposome having an associated US28 or US28 fragment

A variety of cell lines can be used in this aspect of the invention. In one group of embodiments, the cell line is a mouse cell line (e.g., NSO cells from R&D Systems, Minneapolis, Minn., USA). In other embodiments, the cell line is a human cell line (e.g., primary human lung and foreskin fibroblasts from Clonetics, San Diego Calif., USA, or human diploid lung fibroblasts (MRC-5 and WI-38), or HUVECs). Additionally, human embryonic kidney 293 cells (“HEK293” from American Tissue Culture Collection) can also be used. In still other embodiments, the cell line is a primary rhesus monkey dermal fibroblast (from University of California at Davis Primate Center). In each instance, the cell lines described can be infected with whole virus (CMV) or transfected with US28 cDNA, typically under the control of a CMV promoter, using conventional methods. Alternatively, cell-free systems can also be employed wherein a fragment of US28 (e.g., NH₂-terminal peptide, extracellular loops and the like) can be used alone (or in combinations of US28 fragments) to assay binding levels of a chemokine in the presence of a candidate agent. In still other embodiments, expressed or synthesized receptor proteins of US28 can be embedded in artificial membrane systems to assay for chemokine binding in the presence of a candidate agent (see for example, systems described in Kitaguchi, et al., Biochem. Biophys. Res. Commun. 261(3):784-789 (1999) and Myung, et al., Anal. Biochem. 270(2):303-313 (1999)).

For assays using cells, the cells are cultured in a suitable buffer (e.g., IMDM-5% FBS, DMEM 1885-10% FCS, HUVEC complete medium, and the like) then centrifuged and resuspended in assay buffer (e.g., HEPES with NaCl, CaCl₂, MgCl₂, and BSA) to a concentration of from about 5×10⁵ to about 5×10⁷, preferably from about 2 to about 8×10⁶. Aliquots of the cells are then contacted with the candidate compounds and labeled chemokine.

A variety of chemokines can be used in this aspect of the invention, including, for example, fractalkine, RANTES, MCP-3, MIP-1α and MCP-1. A number of the chemokines are commercially available from sources such as R&D Systems or Peprotech, Inc., N.J., USA. Preferably, the labeled chemokine is labeled fractalkine. Additionally, a variety of labels can also be used with the chemokines described above. Typically, the label will be a fluorescence label, a phosphorescence label, a radiolabel, a colorimetric label, or the like. In preferred embodiments the labeled chemokine is a radiolabeled fractalkine, more preferably, ¹²⁵,-fractalkine.

After contacting the cells with one or more candidate compounds in the presence of labeled chemokine, the assay mixture is typically incubated for a period of time of from about 1 to about 6 hours at a temperature of from about 1 to about 10° C. Preferably the mixture is incubated for a period of from about 2 to about 4 hours at a temperature of about 4° C. One of skill in the art will understand that a variety of assay conditions can be employed, depending on the cell line used, the concentrations of the compounds and chemokine and the concentration of the cells themselves.

Following incubation the assay wells can be harvested under vacuum using filter plates, pre-soaked with PEI solution (for those embodiments carried out on 96-, 384-, 1536-well or larger plates). Scintillation fluid (for radiolabel assays) is added, the plates are sealed and the wells are counted. Alternatively, other quantitative methods are employed when, for example, fluorescent labels are used.

B. Compounds Which Block CMV Dissemination

Using the assays described herein, compounds have now been identified which block CMV dissemination.

In one group of embodiments, the compounds have the formula:

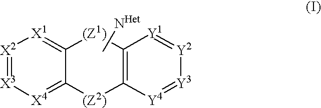

wherein X¹, X², X³ and X⁴ are each independently N or C—R¹, wherein R¹ is H, halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, or di(C₁-C₄)alkylamino. Similarly, Y¹, y², y³ and Y⁴ are each independently N or C-R², wherein R² is H, halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, or di(C₁-C₄)alkylamino.

The symbol Z¹ represents a substituted or unsubstituted (C₁-C₃)alkylene. The symbol Z² represents a divalent moiety selected from —O—, —S—and —N(R)—wherein R is H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, or di(C₁-C₄)alkylamino.

The symbol N^{Het} represents a substituted or unsubstituted 4-, 5-, 6-, or 7-membered nitrogen heterocycle.

In preferred embodiments, at least two of X¹, x², X³ and X⁴ are CH, more preferably three of X¹, X², X³ and X⁴ are CH and the fourth is C-R¹, wherein R¹ is halogen, (C₁-C₄)alkylthio, (C₁-C₄)haloalkyl, (C₁-C₄)haloalkoxy, nitro, cyano, or (C₁-C₄)acyl. Also preferred are those embodiments in which Y¹, Y², Y³ and Y⁴ are each independently C-R², wherein R² is H, halogen, (C₁-C₄)alkyl, (Cl-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, di(C₁-C₄)alkylamino. More preferably, each of Y¹, Y², Y³ and Y⁴ are independently C-R², wherein R² is H, halogen, (C₁-C₄)alkylthio, or (C₁-C₄)haloalkyl.

In other preferred embodiments, Z¹ represents an ethylene or propylene group, more preferably an ethylene group in which N^{Het} is attached at the position adjacent to the ring defined by Y¹, Y², Y³ and Y⁴.

Also preferred are those embodiments in which Z² is —O—or —S—, more preferably —S—.

Preferred groups for N^{Het} are the substituted or unsubstituted 5- or 6-membered nitrogen heterocycles. Particularly preferred heterocycles include piperidine, piperazine, pyrrolidine, oxazoline, imidazoline, pyrazine and morpholine. More preferably, N^{Het} is a substituted or unsubstituted 6-membered nitrogen heterocycle. In the most preferred embodiments, N^{Het} is a substituted or unsubstituted piperazine which is attached to Z¹ through a nitrogen atom of the piperazine ring. Preferred substituents for the piperazine ring are (C₁-C₄)alkyl, (C₁-C₄)haloalkyl, or (C₁-C₄)acyl. Further preferred Substituents are (C₁-C₄)alkyl, with methyl, ethyl and propyl substituents being the most preferred.

In the most preferred embodiments, the compounds are substituted 10-piperazino-10,11 -dihydrodibenzo(b,f)thiepins having the formula:

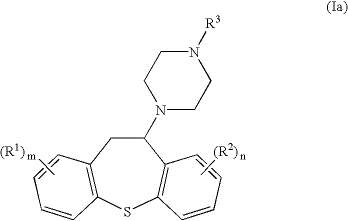

wherein the subscripts m and n are independently integers of from 0 to 3, preferably 0 to 2, more preferably 0 or 1; and R¹ and R² are substituents independently selected from the group of halogen, (C₁-C₄)alkyl, (C₁-C₄)alkoxy, (C₁-C₄)alkylthio, (C₁-C₄)haloalkyl, C₁-C₄)haloalkoxy, nitro, cyano, (C₁-C₄)acyl, amino, (C₁-C₄)alkylamino, and di(C₁-C₄)alkylamino. The symbol R³ represents (C₁-C₄)alkyl, (C₁-C₄)haloalkyl, or (C₁-C₄)acyl.

In particularly preferred embodiments, m is 0 and n is 1. More preferably, m is 0, n is 1 and R² is selected from the group of halogen, (C₁-C₄)alkyl, (C₁-C₄)alkoxy, (C₁-C₄)alkylthio and (C₁-C₄)haloalkyl. Still further preferred are those embodiments in which m is 0, n is 1 and R² is selected from the group of halogen and (C₁-C₄)alkylthio. Most preferably, the R² substituent is at the 8-position of the dihydrodibenzo(b,f)thiepin ring system.

Particularly preferred compounds for use in the present invention are methiothepin (free base or salt, CAS No. 20229-30-5) and octoclothepin (free base or salt, CAS No. 4789-68-8, for the maleate salt).

Other suitable compounds for use in the present invention (compositions and methods) are described in U.S. Pat. No. 3,379,729 “Piperazinyldibenzothiepins” Apr. 23, 1968. See also U.S. Pat. No. 4,444,778. Still other related and useful dihydrodibenzo(b,f)thiepins are described in Jilek, et al., Collect. Czech. Chem. Commun. 33(6):1831-1845 (1968).

C. Compositions Useful in the Treatment of CMV Infection

The present invention also provides compositions useful for preventing CMV dissemination in a host, which comprises a pharmaceutically acceptable carrier or adjuvant and an effective amount of a compound identified using the assays described herein. Preferably, the compound is a compound of formula I, more preferably a compound of formula Ia. Other preferred compounds are those described in Provisional Application Ser. No. ______, filed Aug. 30, 2001 entitled “Bicyclic Compounds as Inhibitors of Chemokine Binding to US 28”, incorporated herein by reference. Particularly preferred compounds are those exemplified in the tables of the noted application.

Typically, the compositions contain from about 0.1% to about 99% by weight of active compound, and preferably from about 10% to about 60% by weight depending on which method of administration is employed.

A CMV dissemination-inhibiting amount is that amount of active compound required to slow the progression of viral dissemination or reduce the amount of viral dissemination from that which would otherwise occur without administration of the compound. Or, it is an amount of active compound required to slow the progression or reduce the intensity of symptoms resulting from CMV infection or elimination thereof.

CMV dissemination-inhibiting activity of compounds of the invention can be determined according to the assays described herein. The assays provide an indication of chemokine binding to US28, more typically fractalkine binding to US28. The compounds provided herein inhibit the binding of fractalkine to US28 with activity expressed as IC50 (that amount of compound that reduces fractalkine binding by 50%). The compounds provided herein will typically exhibit an IC50 of approximately 50 μg/mL or less, preferably 25 μg/mL or less, more preferably 10 μg/mL or less, and most preferably less than 1 μg/mL.

For the compositions of the invention, the proportion of each carrier, diluent or adjuvant is determined by the solubility and chemical nature of the compound and the route of administration according to standard pharmaceutical practice. In order to obtain consistency of administration, however, it is preferred that a composition of the invention is in the form of a unit dose. For example, the unit dose presentation forms for oral administration may be tablets and capsules and may contain conventional excipients such as binding agents (e.g., acacia, gelatin, sorbitol, or polyvinylpyrrolidone), fillers (e.g., lactose, sugar, maize-starch, calcium phosphate, sorbitol or glycine), tableting lubricants (e.g., magnesium stearate), disintegrants (e.g., starch, polyvinylpyrrolidone, sodium starch glycoallate or microcrystalline cellulose), or pharmaceutically acceptable wetting agents (e.g., sodium lauryl sulfate).

The compounds may be injected parenterally; this being intramuscularly, intravenously, or subcutaneously. For parenteral administration, the compound may be used in the form of sterile solutions containing other solutes, for example, sufficient saline or glucose to make the solution isotonic. The amount of active ingredient administered parenterally will be approximately 0.01 to 250 mg/kg/day, preferably about 1 to 10 mg/kg/day, more preferably about 0.5 to 30 mg/kg/day, and more most preferably about 1-20 mg/kg/day.

The compounds may be administered orally in the form of tablets, capsules, or granules containing suitable excipients such as starch, lactose, white sugar and the like. The compounds may be administered orally in the form of solutions which may contain coloring and/or flavoring agents. The compounds may also be administered sublingually in the form of tracheas or lozenges in which each active ingredient is mixed with sugar or corn syrups, flavoring agents and dyes, and then dehydrated sufficiently to make the mixture suitable for pressing into solid form. The amount of active ingredient administered orally will depend on bioavailability of the specific compound.

The solid oral compositions may be prepared by conventional methods of blending, filling, tableting, or the like. Repeated blending operations may be used to distribute the active agent throughout those compositions employing large quantities of tillers. Such operations are, of course, conventional in the art. The tablets may be coated according to methods well known in normal pharmaceutical practice, in particular with an enteric coating.

Oral liquid preparations may be in the form of emulsions, syrups, or elixirs, or may be presented as a dry product for reconstitution with water or other suitable vehicle before use. Such liquid preparations may or may not contain conventional additives. For example suspending agents, such as sorbitol, syrup, methyl cellulose, gelatin, hydroxyethylcellulose, carboxymethylcellulose, aluminum stearate gel, or hydrogenated edible fats; emulsifying agents, such as sorbitan monooleate or acaci; non-aqueous vehicles (which may include edible oils), such as almond oil, fractionated coconut oil, oily esters selected from the group consisting of glycerin, propylene glycol, ethylene glycol, and ethyl alcohol; preservatives, for instance methyl para-hydroxybenzoate, ethyl para-hydroxybenzoate, n-propyl parahydroxybenzoate, or n-butyl parahydroxybenzoate of sorbic acid; and, if desired, conventional flavoring or coloring agents.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. As used herein, topical application is also meant to include the use of mouth washes and gargles.

In another embodiment, the invention provides the subject compounds in the form of a pro-drug, which can be metabolically or chemically converted to the subject compound by the recipient host. A wide variety of pro-drug derivatives are known in the art such as those that rely on hydrolytic cleavage or oxidative activation of the prodrug.

The compositions may be advantageously combined and/or used in combination with other antiviral agents which are either therapeutic or prophylactic agents, and different from the subject compounds. The compositions may also be advantageously combined and/or used in combination with agents that treat or induce conditions often associated with the viral infections that are sensitive to the present compounds, such as anti-HIV agents or immunosuppressive agents. In many instances, administration in conjunction with the subject compositions enhances the efficacy of such agents. Exemplary antiviral agents include ganciclovir, foscarnet and cidofovir. Exemplary anti-HIV agents include indinavir, ritonavir, AZT, lamivudine and saquinavir. Exemplary immunosuppressive agents include cyclosporin and FK-506. The compositions may also be advantageously used as antiviral prophylactic treatment in combination with immunosuppressive protocols such as bone-marrow destruction (either by radiation or chemotherapy).

D. Methods of Treating CMV Infection

In yet another aspect, the present invention provides novel methods for the use of the foregoing compounds and compositions. In particular, the invention provides novel methods for treating or preventing viral dissemination from CMV infection. The methods typically involve administering to a patient an effective formulation of one or more of the subject compositions.

The invention provides methods of using the subject compounds and compositions to treat disease or provide medicinal prophylaxis to individuals who possess a compromised immune system or are expected to suffer immunosuppressed conditions, such as patients prior to undergoing immunosuppressive therapy in connection with organ transplantation or anticancer chemotherapy. These methods generally involve administering to the host an effective amount of the subject compounds or pharmaceutically acceptable compositions.

The compositions and compounds of the invention and the pharmaceutically acceptable salts thereof can be administered in any effective way such as via oral, parenteral or topical routes. Generally, the compounds are administered in dosages ranging from about 2 mg up to about 2,000 mg per day, although variations will necessarily occur depending on the disease target, the patient, and the route of administration. Preferred dosages are administered orally in the range of about 0.05 mg/kg to about 20 mg/kg, more preferably in the range of about 0.05 mg/kg to about 2 mg/kg, most preferably in the range of about 0.05 mg/kg to about 0.2 mg per kg of body weight per day.

Therapeutic and prophylactic methods of this invention comprise the step of treating patients in a pharmaceutically acceptable manner with those compounds or compositions. Such compositions may be in the form of tablets, capsules, caplets, powders, granules, lozenges, suppositories, reconstitutable powders, or liquid preparations, such as oral or sterile parenteral solutions or suspensions. Compounds of the invention may also be administered via an intraocular implant for treating retinitis as a result of CMV infection. In particular, compounds may be embedded in a polymer based implant which will be release into the eye over an extended period of time.

Physicians will determine the dosage of the present therapeutic agents which will be most suitable. Dosages may vary with the mode of administration and the particular compound chosen. In addition, the dosage may vary with the particular patient under treatment. The dosage of the compound used in the treatment will vary, depending on viral load, the weight of the patient, the relative efficacy of the compound and the judgment of the treating physician. Such therapy may extend for several weeks or months, in an intermittent or uninterrupted manner.

To further assist in understanding the present invention, the following non-limiting examples are provided.

### EXAMPLES

### Example 1

The US28 expressing cells used in most assays consist of a mouse cell line (NSO cells from ATCC) stably expressing transfected US28 cDNA under the control of a CMV promoter (from R & D Systems). These cells were cultured in IMDM-5% FBS, and harvested when the concentration was between 0.5-1.0×10⁶ cells/mL. Some assays were performed with adherent human 293 cells (US28-293 cells) or membranes. The cells were centrifuged and resuspended in assay buffer (20 mM HEPES, 140 mM NaCl, 1 mM CaCl₂, 5 mM MgCl₂, and with 0.2% bovine serum albumin) to a concentration of 5.6×10⁶ cells/mL. Using the Multi-Probe automated system, set up with 8 assay plates at a time, first 0.09 mL of cells was added to the assay plates containing the compounds. The final concentration of the compounds was 5 μg/mL each. Then 0.09 mL of ¹²⁵¹I-fractalkine diluted in assay buffer (final concentration ˜2-10fM, with ˜30,000 cpm per well) was added, the plates sealed and incubated for approximately 3 hours at 4 degrees C on a shaker platform. The assay plates were harvested using Packard filter plates, pre-soaked in PEI solution, on the vacuum harvest apparatus. Scintillation fluid (35 μL) was added to all wells, the plates were sealed and counted in a Top Count scintillation counter. Control wells containing either diluent only (for total counts) or excess Fractalkine (1 μg/mL, for non-specific binding) were used to calculate the percent of total inhibition for each set of compounds. Further tests on individual compounds were carried out in the same manner.

### Example 2

As secondary assays for compounds that specifically inhibited the binding of radiolabled Fractalkine to US28, cytoplasmic calcium mobilization experiments were done by loading US28-293 cells with INDO-1 dye (45 min. at room temperature), washing with PBS, and resuspending into Ca2 +‘flux’ buffer (HBSS with 1% fetal bovine serum). For each test, 1×10⁶ cells were incubated at 37° C. in the cuvette of a PTI spectrometer, and the ratio of 410/490 nm emission plotted over time (typically 2-3 minutes), with compounds added at 5 seconds, followed by fractalkine at 60 seconds. A rise in intracellular Ca2+ is typically seen when US28-293 cells are challenged with fractalkine, an indication that the US28 receptor bound to the ligand, engaged a G-protein linked cascade which resulted in the mobilization of Ca2+ in the cytoplasm of the US28-bearing cells. Compounds which inhibited fractalkine binding were tested in this assay for the effects on Ca2+ in this system.

### Example 3

This example illustrates the effects of octoclothepin and methiothepin at inhibiting the binding of fractalkine to US28.

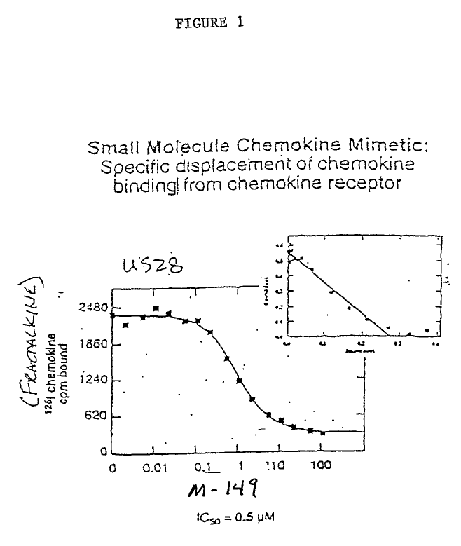

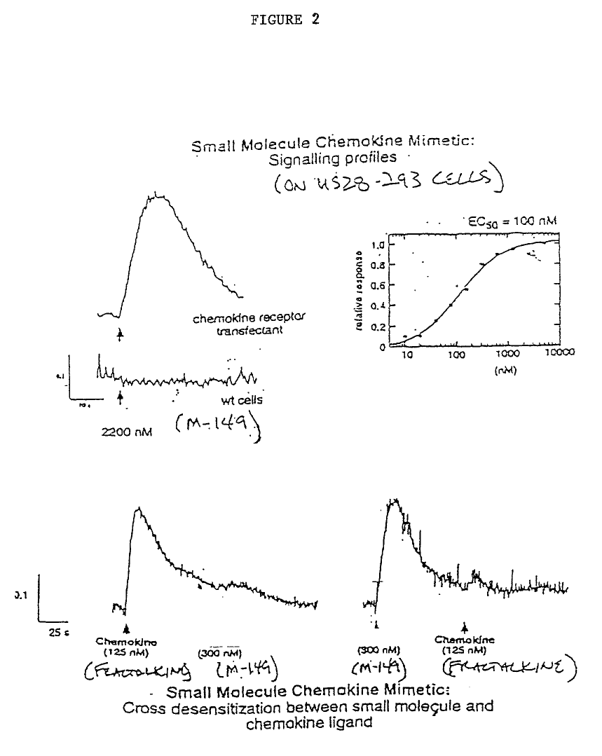

Methiothepin mesylate (from the RBI division of Sigma Chemical Co., St. Louis, Mo., USA, Catalog No. M-149) and octoclothepin maleate (from RBI, Catalog No. O-111) were evaluated in the assays described in Examples 1 and 2. A dose response of methiothepin mesylate and octoclothepin maleate against fractalkine on US28-NSO cells is shown in FIG. 1. The IC₅₀ values were 0.3 μM for methiothepin mesylate and 0.7 μM for octoclothepin maleate. Additionally, when the compounds were tested for calcium mobilization in US28-293 cells, both compounds were found to act as competitive agonists for the US28 receptor, mimicking the action of fractalkine in both binding and signaling (see FIG. 2).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An assay for identifying a compound useful for blocking CMV dissemination is a host, comprising the step of determining whether said compound inhibits the binding of a chemokine to US28 or a US28 fragment.

2. An assay in accordance with claim 1, wherein said chemokine is selected from the group consisting of fractalkine, MIP-1α, MIP-1β, MCP-1 and RANTES.

3. An assay in accordance with claim 1, wherein said chemokine is fractalkine.

4. An assay in accordance with claim 1, wherein said step of determining comprises specifically binding labeled fractalkine to the ligand binding domain of US28.

5. A method for preventing dissemination of CMV in a human, comprising administering an effective amount of a compound which blocks the binding of a chemokine to US28 or a US28 fragment.

6. A method in accordance with claim 5, wherein said compound was identified by the assay of claim 1.

7. A method in accordance with claim 5, wherein said compound has the formula: 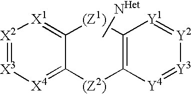
wherein
X¹, X², X³ and X⁴ are each independently members selected from the group consisting of N and C—R¹, wherein R¹ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Y¹, Y², Y³ and Y⁴ are each independently members selected from the group consisting of N and C—R², wherein R² is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Z¹ is a divalent moiety selected from the group consisting of (C₁-C₃)alkylene;
Z² is a divalent moiety selected from the group consisting of —O—, —S— and —N(R³)— wherein R³ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and
N^{Het} is a substituted or unsubstituted 4-, 5-, 6-, or 7-membered nitrogen heterocycle.

8. A method in accordance with claim 7, wherein X¹, X³, X⁴, Y¹, Y², Y³ and Y⁴ are all CH; Z² is —S—, and N^{Het} is a substituted 6-membered nitrogen heterocycle.

9. A method in accordance with claim 5, wherein said compound has the formula: 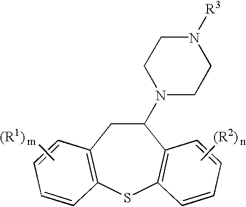
wherein
the subscripts m and n are independently integers of from 0 to 3;
R¹ and R² are substituents independently selected from the group consisting of halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and
R³ is a substituent selected from the group consisting of (C₁-C₄)alkyl, (C₁-C₄) haloalkyl and (C₁-C₄)acyl.

10. A method in accordance with claim 9, wherein m is 0 and n is 1.

11. A method in accordance with claim 9, wherein m is 0, n is 1 and R² is selected from the group consisting of halogen, (C₁-C₄)alkyl, (C₁-C₄)alkoxy, (C₁-C₄)alkylthio and (C₁-C₄)haloalkyl.

12. A method in accordance with claim 9, wherein m is 0, n is 1 and R² is selected from the group consisting of halogen and (C₁-C₄)alkylthio.

13. A method in accordance with claim 5, wherein said compound is selected from the group consisting of methiothepin, octoclothepin and pharmaceutically acceptable salts thereof.

14. A method for reducing cell motility in a CMV-infected cell, said method comprising contacting said CMV-infected cell with a motility-reducing amount of a compound that inhibits chemokine binding to US28 on the surface of said infected cell.

15. A method in accordance with claim 14, wherein said chemokine is a member selected from the group consisting of fractalkine, MIP-1α, MIP-1β, MCP-1 and RANTES.

16. A method in accordance with claim 14, wherein said chemokine is fractalkine.

17. A method in accordance with claim 14, wherein said compound has the formula: 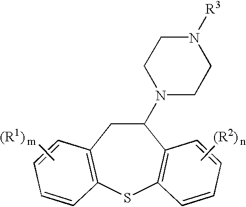
wherein
the subscripts m and n are independently integers of from 0 to 3;
R¹ and R² are substituents independently selected from the group consisting of halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and
R³ is a substituent selected from the group consisting of (C₁-C₄)alkyl, (C₁-C₄)haloalkyl and (C₁-C₄)acyl.

18. A method in accordance with claim 17, wherein m is 0 and n is 1.

19. A method in accordance with claim 17, wherein m is 0, n is 1 and R² is selected from the group consisting of halogen, (C₁-C₄)alkyl, (C₁-C₄)alkoxy, (C₁-C₄)alkylthio and (C₁-C₄)haloalkyl.

20. A method in accordance with claim 17, wherein m is 0, n is 1 and R² is selected from the group consisting of halogen and (C₁-C₄)alkylthio.

21. A method in accordance with claim 14, wherein said compound is selected from the group consisting of methiothepin, octoclothepin and pharmaceutically acceptable salts thereof.

22. A pharmaceutical composition comprising a pharmaceutically acceptable carrier and a compound of the formula: 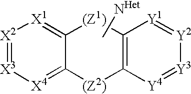
wherein
X¹, X², X³ and X⁴ are each independently members selected from the group consisting of N and C—R², wherein R² is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Y¹, Y², Y³ and Y⁴ are each independently members selected from the group consisting of N and C—R², wherein R² is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Z¹ is a divalent moiety selected from the group consisting of (C₁-C₃)alkylene;
Z² is a divalent moiety selected from the group consisting of —O—, —S— and —N(R³)— wherein R³ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and
N^{Het} is a substituted or unsubstituted 4-, 5-, 6-, or 7-membered nitrogen heterocycle.

23. A composition in accordance with claim 22, wherein X¹, X³, X⁴, Y¹, Y², Y³ and Y⁴ are all CH; Z² is —S—, and N^{Het} is a substituted 6-membered nitrogen heterocycle.

24. A composition in accordance with claim 22, wherein said compound has the formula: 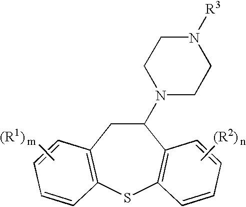
wherein
the subscripts m and n are independently integers of from 0 to 3;
R¹ and R² are substituents independently selected from the group consisting of halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and
R³ is a substituent selected from the group consisting of (C₁-C₄)alkyl, (C₁-C₄)haloalkyl and (C₁-C₄)acyl.

25. A composition in accordance with claim 24, wherein m is 0 and n is 1.

26. A composition in accordance with claim 24, wherein m is 0, n is 1 and R² is selected from the group consisting of halogen, (C₁-C₄)alkyl, (C₁-C₄)alkoxy, (C₁-C₄)haloalkyl and (C₁-C₄)alkylthio.

27. A composition in accordance with claim 24, wherein m is 0, n is 1 and R² is selected from the group consisting of halogen and (C₁-C₄)alkylthio.

28. A composition in accordance with claim 24, wherein said compound is selected from the group consisting of methiothepin, octoclothepin and pharmaceutically acceptable salts thereof.
